(19) United States
(12) Patent Application Publication
(10) Pub. No.: US 2007/0043321 A1
(43) Pub. Date: Feb. 22, 2007

(54) Safe injection device

(76) Inventor: Cheh Tai, Zhongli City (TW)

Correspondence Address: ROSENBERG, KLEIN & LEE, SUITE 101, 3458 ELLICOTT CENTER DRIVE, ELLICOTT CITY, MD, 21043, US

(21) Appl. No.: 11/311,257

(22) Filed: Dec. 20, 2005

Related U.S. Application Data
(62) Division of application No. 11/244,222, filed on Oct. 6, 2005.

(30) Foreign Application Priority Data
Jul. 22, 2005 (TW) 094124843

(51) Int. Cl. A61M 5/00

(52) U.S. Cl. 604/110; 604/187

(57) ABSTRACT

The present invention discloses a safe injector. The safe injector comprises a sleeve, a needle, and push rod, wherein the needle can be hided in the sleeve. The needle can be pushed out of the sleeve for use and be hided in the sleeve again after finishing the use.

### BACKGROUND OF THE INVENTION

1. Field of the Invention

The present invention generally relates to a safe injection device, and more particularly to an injection device with capacity for hiding the needle into the sleeve.

2. Description of the Prior Art

For injecting the drug, the drug is sucked into the syringe and then injected into the patient. The FIG. 1 is a diagram of a normal syringe. The needle is configured on the head of the syringe, and it can be replaced or removed from danger in some designs.

As the syringe needle contacts the blood, it would carry the pathogens and become the medium of the pathogens. Therefore the safekeeping of the syringe should be take care. A cap of the needle is often used for preventing accident. However, the needle could penetrate the fingers when the cap is caped on the needle. Obviously, the manner is very dangerous.

Moreover, sometimes the syringe is intended to be used once. That is, the syringe should be imposable in case. Although the syringe is thrown away after being used, there is no guarantee that the syringe can be reused. The potential danger still exists.

Therefore a new design for more safe usage is needed. Besides, the design should limit the syringe for once usage only.

### SUMMARY OF THE INVENTION

Therefore, in accordance with the previous summary, objects, features and advantages of the present disclosure will become apparent to one skilled in the art from the subsequent description and the appended claims taken in conjunction with the accompanying drawings.

One objective of the present invention is to provide a safe injection device for pulling the needle into the sleeve.

Another object of the present invention is to provide a limited structure for once usage, whereby said safe injection device can not be reusable.

Accordingly, the present provides a safe injection device, comprising a sleeve, pushing rod, and a needle, wherein the needle can be hided in the sleeve. When the safe injection device is used, the needle can be pushed to protruding to the sleeve. After the safe injection device is used, the needle can be hided into the sleeve again. The present invention further comprises a hook inside the sleeve and a fin of the pushing rod for fastening the pushing rod in the sleeve for preventing second usage.

Another side of said sleeve opposite to the needle comprises a cap with an opening, wherein the shape of the opening is used for limiting the movement of the pushing pod. The movement is a lengthwise movement parallel to said channel rather than a widthwise rotation perpendicular to said channel. Therefore each drug can be assigned to a specific shape of the rod. Only when the drug and the shape of the rod match, the drug can get the permission to be injected.

### BRIEF DESCRIPTION OF THE DRAWINGS

The accompanying drawings incorporated in and forming a part of the specification illustrate several aspects of the present invention, and together with the description serve to explain the principles of the disclosure. In the drawings:

FIG. 1 is a diagram illustrates the prior art;

FIG. 2A through FIG. 2G are diagrams depicts one embodiment of the present invention;

FIG. 3A through FIG. 3C are diagrams shows another embodiment of the present invention;

FIG. 4A through FIG. 4D are diagrams shows the structure of one embodiment of the present invention;

FIG. 5 is a diagram shows the structure of one embodiment of the present invention;

FIG. 6A is a diagram shows the structure of one embodiment of the present invention;

FIG. 6B is diagram shows the operation example of one embodiment of the present invention;

FIG. 7A through FIG. 7C are diagrams shows the structure and the operation example of one embodiment of the present invention;

FIG. 8A through FIG. 8B are diagrams shows the structure and the operation example of one embodiment of the present invention; and

FIG. 9A through FIG. 9F are diagrams shows the structure and the operation example of one embodiment of the present invention.

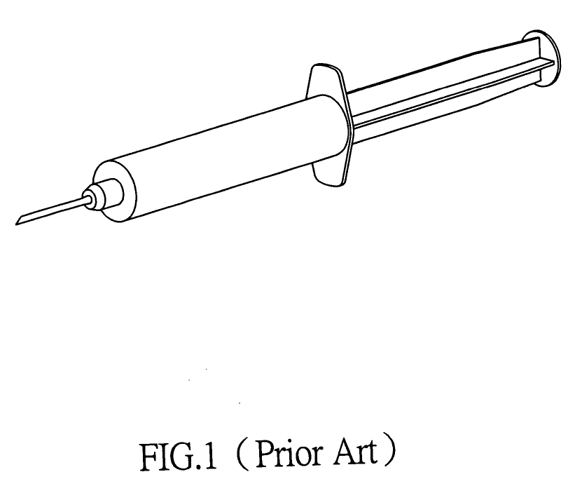

### DETAILED DESCRIPTION OF THE PREFERRED EMBODIMENTS

The present disclosure can be described by the embodiments given below. It is understood, however, that the embodiments below are not necessarily limitations to the present disclosure, but are used to a typical implementation of the invention.

Having summarized various aspects of the present invention, reference will now be made in detail to the description of the invention as illustrated in the drawings. While the invention will be described in connection with these drawings, there is no intent to limit it to the embodiment or embodiments disclosed therein. On the contrary the intent is to cover all alternatives, modifications and equivalents included within the spirit and scope of the invention as defined by the appended claims.

It is noted that the drawings presents herein have been provided to illustrate certain features and aspects of embodiments of the invention. It will be appreciated from the description provided herein that a variety of alternative embodiments and implementations may be realized, consistent with the scope and spirit of the present invention.

It is also noted that the drawings presents herein are not consistent with the same scale. Some scales of some components are not proportional to the scales of other components in order to provide comprehensive descriptions and emphasizes to this present invention.

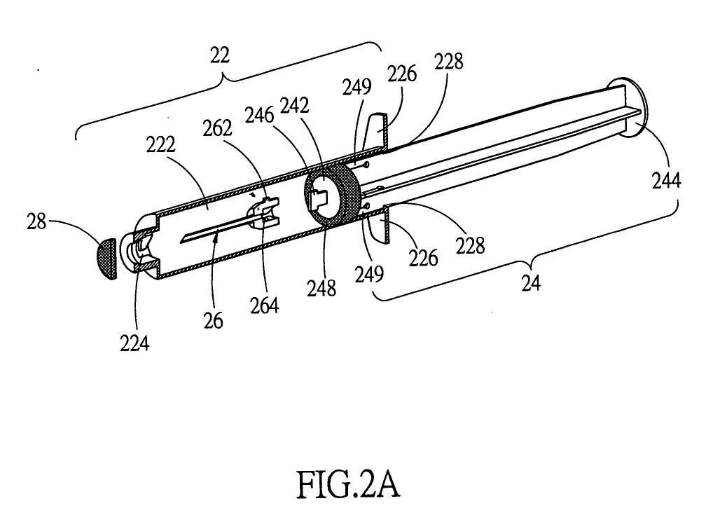

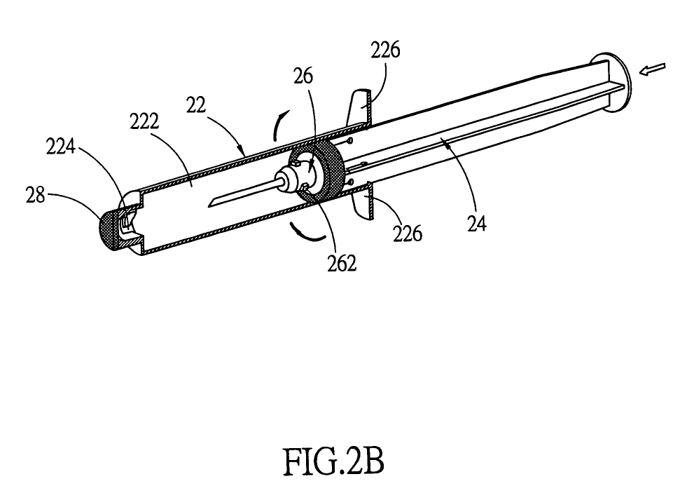

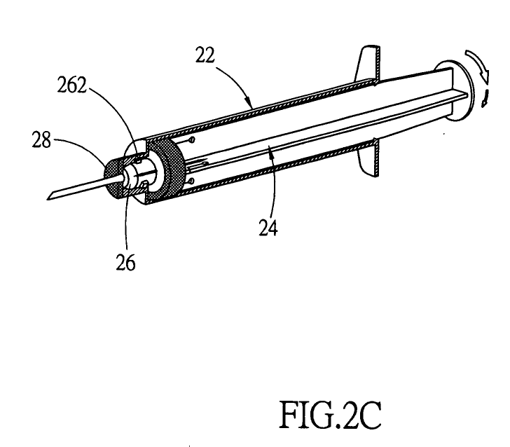

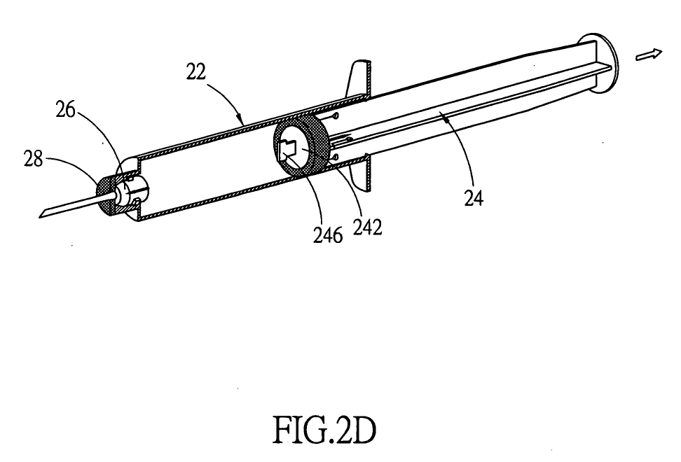

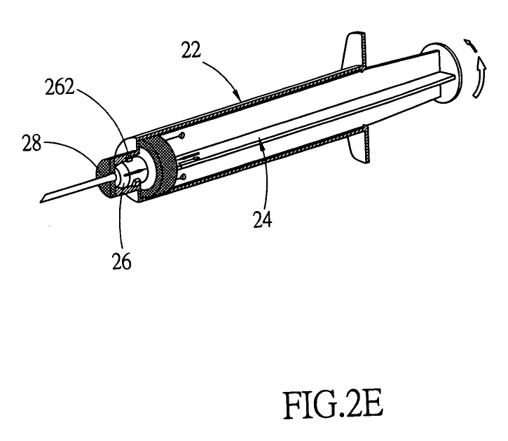

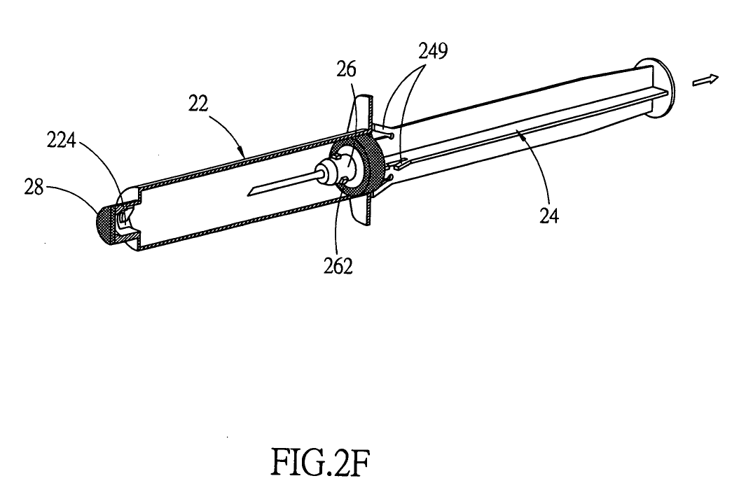

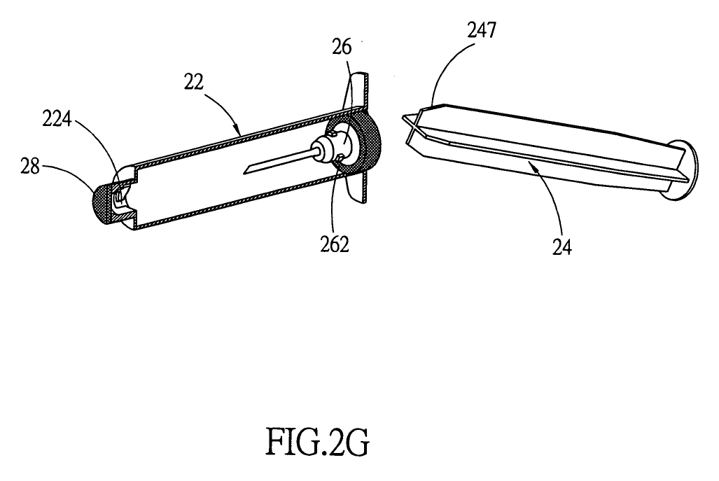

One embodiment of the present invention is illustrated in FIG. 2A, which is a safe injection device, comprising a sleeve 22, a needle 26, and a push rod 24. The sleeve includes a channel 222, a first fastener 224, and a finger holder 226. Both sides of the channel 222 are a first opening and a second opening separately. The first fastener 224 is inside the first opening and the finger holder 226 is outside the second opening.

Besides, push rod 24 includes a second fastener 246 and a flexible material 248. The both side of the push rod 24 are a first end 242 and a second end 244, wherein the second fastener 246 locates on the first end 242 and the first end 242 is covered by the flexible material 248. The sectional profile of the flexible material 248 matches the sectional profile of the channel 222 for waterproof, referring to FIG. 2A. When the first end 242 is plugged into the channel 222, the flexible material 248 can nestle up against the inner wall of the channel 222, therefore the liquid or air will not (be easy to) leak out from the first end 242.

The needle 26 includes a third fastener 262 and a fourth fastener 264. The third fastener 262 and the fourth fastener 264 are used to connect the first fastener 224 and the second fastener 246. By rotating in a first direction, such as the clockwise direction, the first fastener 224 can connect to the third fastener 262 and the second fastener 246 can disconnect from the fourth fastener 264, referring to FIG. 2B and FIG. 2C. Referring to FIG. 2D, when the push rod 24 is pulled out, the needle 26 is left in the first opening. Besides the inner pressure in the channel 222 is pulled down, the external liquid or air will be sucked into the channel 222 via the needle 26. Contrary to the first direction, by rotating in a second direction, such as the count clock wise direction, the first fastener 224 can disconnect from the third fastener 262 and the second fastener 246 can connect to the fourth fastener 264, referring to FIG. 2E.

Furthermore, the embodiment can comprise a cover 28 for sealing the first opening, wherein the cover 28 can be penetrated by the needle 26, referring to FIG. 2F. The cover 28 is formed by a flexible material. Therefore even it is penetrated by the needle 26, the cover 28 and the needle 26 can still keep water proof. Moreover, the cover 28 can further include a disinfection cavity (not shown in the figure), which is filled with disinfection liquid. Therefore the needle 26 will go through the disinfection liquid when it penetrates the cover 28. Besides, the disinfection liquid can flow out to disinfect the skin toughed by the needle 26 because of the breakage of the cavity penetrated by the needle 26.

In the embodiment, the needle 26 can include at least one hook 228, referring to FIG. 2A. When the first end 242 is pulled toward the second opening, the hook 228 will seize the flexible material 248 for stopping the first end 242 to leave the channel 222, wherein the first end 242 is pushed into the channel 222 against the edge of the hook 228. Moreover the push pod 24 in the embodiment can further comprise at least a flexible fin 249, referring to FIG. 2F. The flexible fin 249 is pushed toward the push pod 24 when the first end 242 is pushed into the second opening. And the flexible fin 249 spreads when it is pulled out of the second opening. Thus movement of the first end 242 will be limited by the flexible fin 249 and the hook 228 when the flexible fin 249 is pulled out of the second opening, referring to FIG. 2F. Furthermore, push pod 24 can further comprise at least one indentation 247 for providing an easy way to break the push pod 24 at the indentation 247, referring to FIG. 2G.

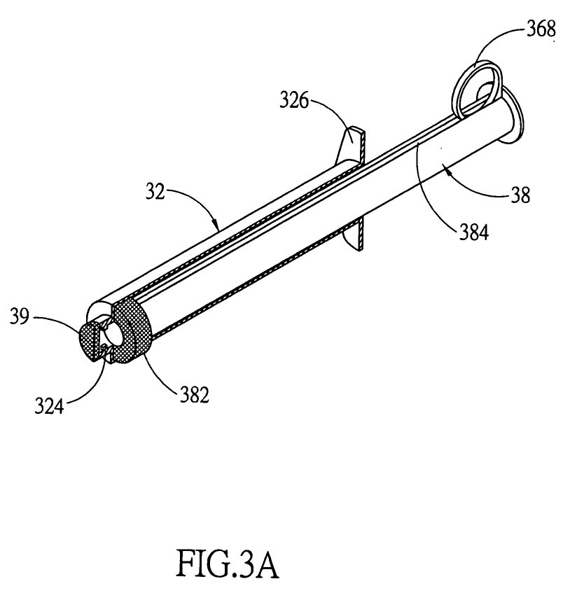

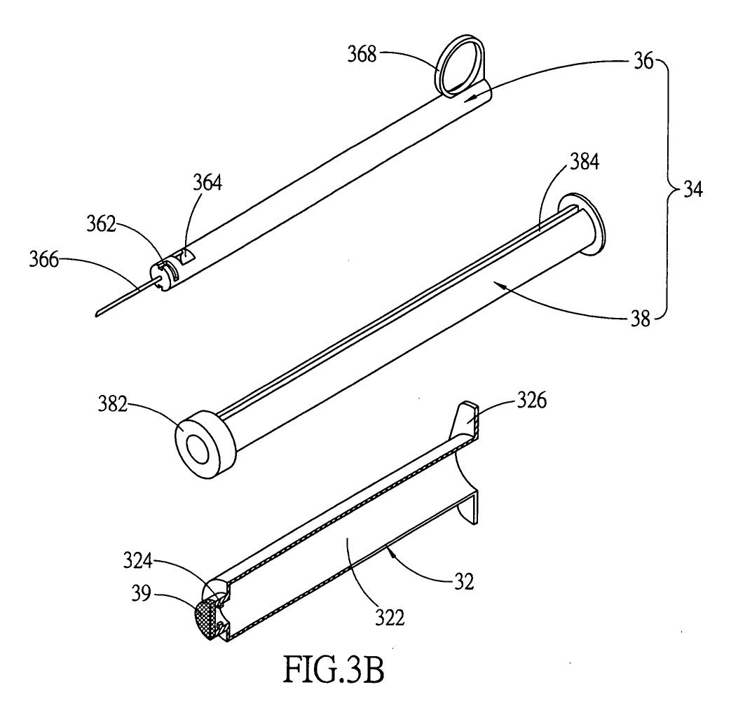

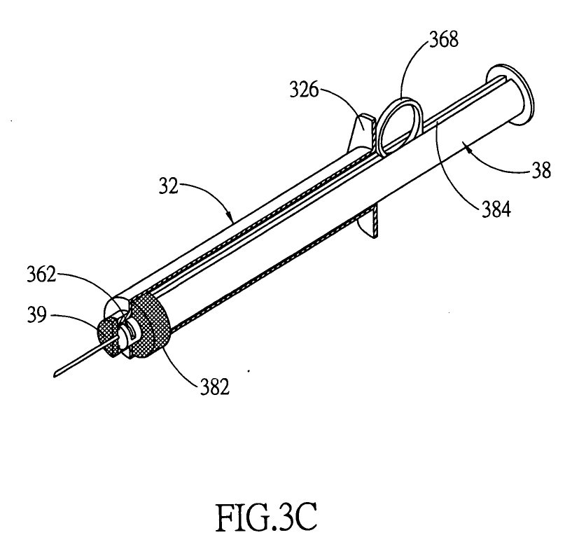

Another embodiment of the present invention is a safe injecting device, referring to FIG. 3A to FIG. 3C, including a sleeve 32, connecting rod 34, and a cover 36. The sleeve 32 includes a channel 322, a first fastener 324 and a finger holder 326. The both sides of the channel are a first opening and a second opening separately. The first fastener 324 locates within the first opening, and the finger holder 326 locates out of the second opening. The cover 39 is used for sealing the first opening and is available to be penetrated by the needle 366. The cover is formed by a flexible material. Therefore even the cover 39 is penetrated by the needle 366, which and the cover 39 can keep water proof. Besides, the cover 39 can further comprise a disinfection cavity (not shown on the figure), which is filled with a disinfection liquid. While the needle 366 is penetrating the cover 39, it passes through the disinfection liquid, too. Besides, the disinfection liquid can flow out to disinfect the skin toughed by the needle 366 because of the breakage of the cavity penetrated by the needle 366.

Furthermore, the connecting rod 34 comprises a first rod 36 and second rod 38. The both sides of the first rod 36 are a first end and a second end. The first rod 36 also comprises a second fastener 362, an injection opening 364, a needle 366 and a holder 368. The second fastener 362 locates on the first end, and the injection opening 364 locates on the side of the first end. The needle 366 is fastened on the top of the first end and there is a channel between the needle 366 and the injection opening 364. The holder 368 is fastened on the side of the second rod. The connection of the first fastener 324 and the second fastener 362 is made by rotating the connecting rod 34 in a first rotating direction (i.e. the clockwise direction). The disconnection of the first fastener 324 and the second fastener 362 is made by rotating the connecting rod 34 in a second direction that is opposite to the first direction (i.e. the counter clockwise direction).

Moreover, the both sides of the second rod 38 are a third end and a fourth end separately. The second rod 38 comprises a flexible material 382 and a lengthwise opening 384, wherein the flexible material cover the periphery of the third end. The section profile of the first end covered with the flexible material matches the section profile of the channel 322 for water proof. Besides, the lengthwise opening 384 is parallel to the channel 322. The second is limited within the second rod 38, and the holder 368 is limited to move within the lengthwise opening 384.

Accordingly, the first rod 36 can be pushed to the first opening for protruding the needle out of the sleeve 32, referring to FIG. 3C. By pulling out the second rod 38 for sucking the liquid or the air into the channel 322 via the needle 366, wherein the liquid or the air is sucked by the needle 366 and flow into the channel 322 through the injection opening 364.

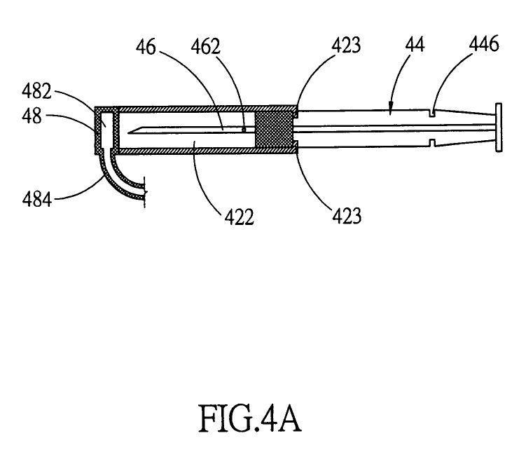

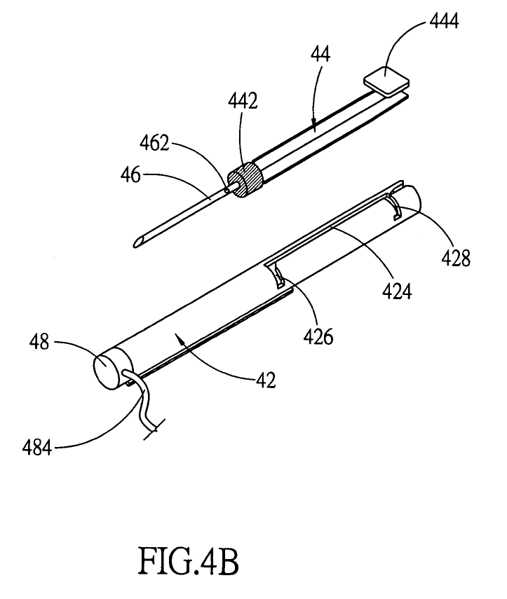

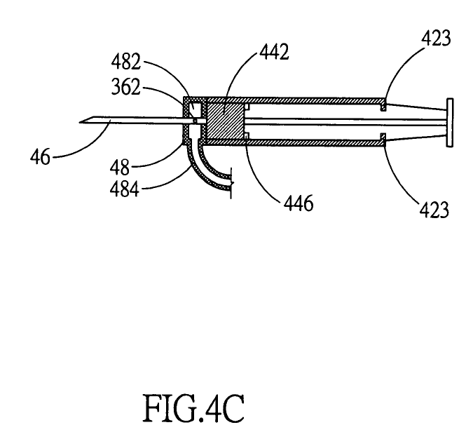

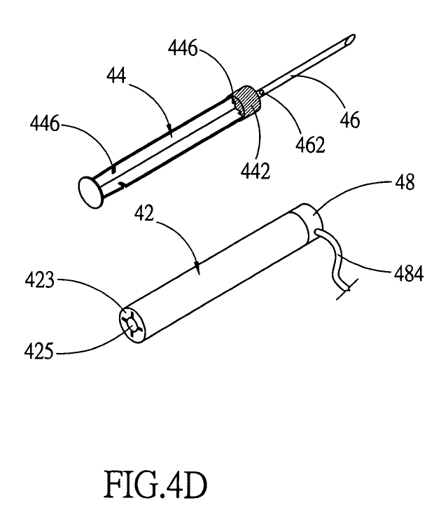

A further embodiment of the present invention is a safe injection device, referring to FIG. 4A and FIG. 4B. The safe injection device comprises a sleeve 42, a push pod 44, a needle 46, and a cover 48. The sleeve 42 includes a channel 422. The push pod 44 includes a flexible material 442. Both sides of the push pod 44 are a first end and a second end, wherein the first end is limited within the channel 422 and the flexible material 442 covers the first end. The section profile of the first end covered by the flexible material 442 matches the section profile of the channel 422 for water proof.

Besides, an opening 462 locates on the side of the needle 46, and the needle 46 locates the top of the first end. Moreover, the forgoing cover 48 seals the first opening. Besides, the cover 48 includes a cavity 482 and a pipe 484, the pipe 484 connects to the cavity. Referring to FIG. 4C, when the first end is pushed to reach the cover 48, the cover 48 is penetrated by the needle 46 and the opening 462 is within the cavity 482 at the moment.

Furthermore, referring to 4D, the sleeve 42 can further include a cap 423 with an opening 425. The shape of the opening 425 is used to limit the movement of the body of the sleeve 42, thus the body of the sleeve 42 can only move within the opening 425 parallel to the channel 422 and can not rotate within the opening 425. In an example of the embodiment, the push rod 44 can further comprise at least one widthways gap 446. Therefore the push rod 44 can be rotated only when the widthways gap 446 crosses the opening 425, whereby the body of the push rod 44 can get stuck with the cap 423 for preventing from moving. The push pod 44 can comprise a plurality of gap 446.

In another example of the embodiment, referring to FIG. 4B, the forgoing sleeve 42 can further comprise a lengthwise opening 424, wherein the lengthwise opening 424 is parallel to the channel 422. The push pod 44 can further comprise a holding means 444, the holding means 444 locates on the side of the second end, wherein the holding means 444 is limited to move within the lengthwise opening 424. Besides, the both ends of the lengthwise opening 424 can include gap 426 and gap 428 separately. The holding means 444 can be push into the gap 426 or gap 428 for preventing from moving.

Furthermore, the forgoing channel 422 can further comprise at least one hook (not shown) toward to the first opening. When the first end is pulled out toward the second opening, the first end will get stuck with the flexible material 442 for preventing the first end from leaving the channel 422. The first end is jostled with the edge of the hook 421 to be pushed into the channel 422.

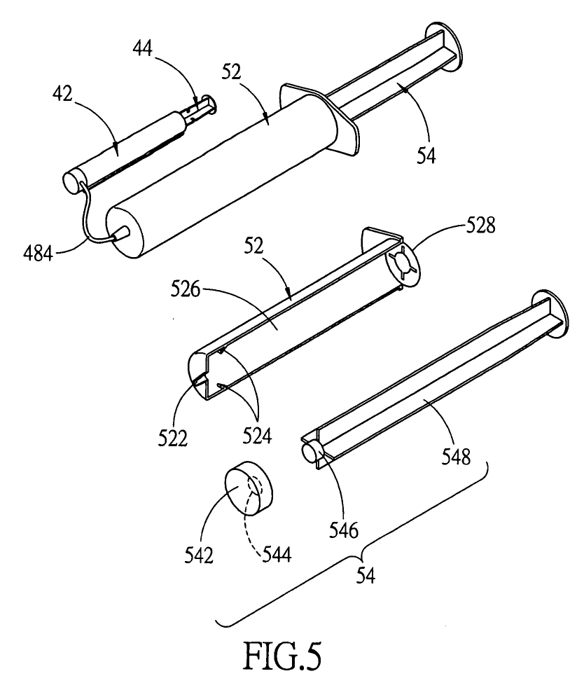

Moreover, referring to FIG. 5, the embodiment can further include an injecting means, comprising a sleeve 52 and a push pod 54. The sleeve 52 includes a nozzle 522 and a channel 526, wherein the nozzle 522 locates one end of the channel 526. The pipe 484 connects to the nozzle 522. Besides, one end of the push pod 54 is a flexible material 542, which coincide with the inner wall of the channel 526 for water proof. Furthermore, the channel 526 of the injecting means further comprise at least one hook 524 which is inward the nozzle 522. When the flexible material 542 is pulled, the flexible material 542 will be stopped by the hook 524 for preventing the flexible material 542 from leaving the channel. The flexible material 542 is jostled with the edge of the hook 524 to be pushed into the channel 526. Moreover, one end of the sleeve 52 which is opposite to the nozzle 522 can further comprise a cap 528 with an opening which has a geometric shape. The geometric shape of the opening is used to limit to the movement of the push rod 54 within the channel 526, wherein the section profile of the push rod 54 which is perpendicular to the channel 526 must match the geometric shape of the opening. Besides, the push rod 54 can be formed by a flexible material 542 and a rod 548 which have a first fastener 544 and a second fastener 546 separately. The flexible material 542 connects to the rod 548 by connecting the first fastener 544 and the fastener 546. The flexible material 542 and a drug which is correspondent to a specific push rod 548 can be pre-configured within the sleeve 52. The geometric shape of the opening is used to confirm whether the rod 548 is correspondent to the drug or not. That is, the wrong rod 548 which is not correspondent to the drug pre-configured within the sleeve 52 can not be used to inject the drug.

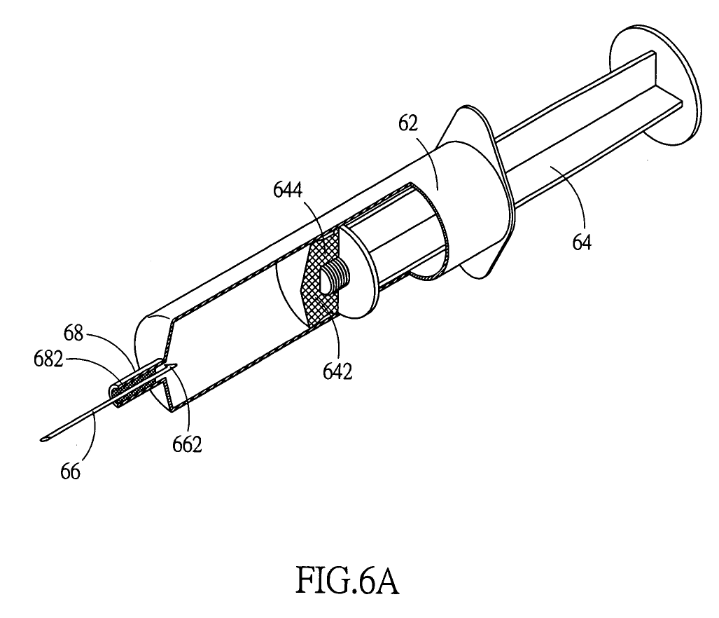

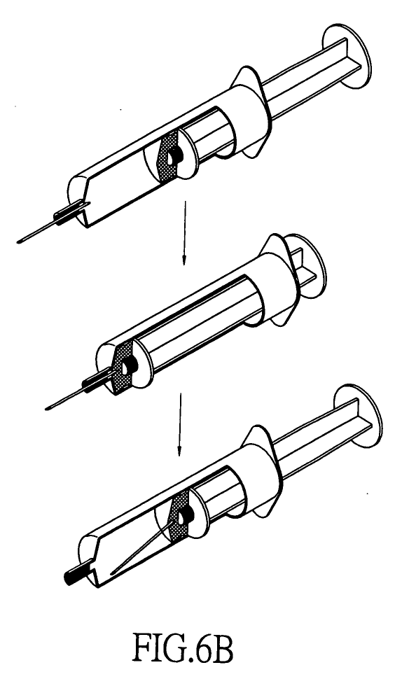

FIG. 6A is a diagram illustrating one embodiment of the present invention, which comprises a sleeve 62, a push rod 64, a needle 66 and a needle base 68. The sleeve 62 comprises a channel whose both ends are a first end and a second end, wherein the first end is connected with the needle base 68. The needle 66 is configured on the needle base 68. The needle base 68 includes a first flexible material 682 within it for tightly holding the needle 66. Thus the needle 66 penetrates the needle base 68, whereby one end of the needle 66 is outside the sleeve 62 and the other end of the needle 66 is inside the sleeve 62. The both ends of the push rod 64 are a third end and a fourth end separately. The third end is pushed into the sleeve 62 via the second end. The third end is covered with a second flexible material 642, wherein the section profile of the flexible material 642 matches the section profile of the channel for water proof. The connection of the first end and the flexible material 642 can be a pluggable connection by a fastener means. The pluggable connection of the fastener means can be a bolt connection, a locking pawl connection, tightly holding connection.

The end of the needle 66 inside the sleeve 62 comprises at least a hook 662 protruding within the channel. Referring to FIG. 6B, the hook 662 penetrate into the second flexible material 642 when the push rod 64 is pushed forward. With the connection of the needle 66 and the hook 662, then the needle can be completely pulled into the sleeve 62 by pulling the push rod 64 for receiving the needle 66 within the sleeve 62.

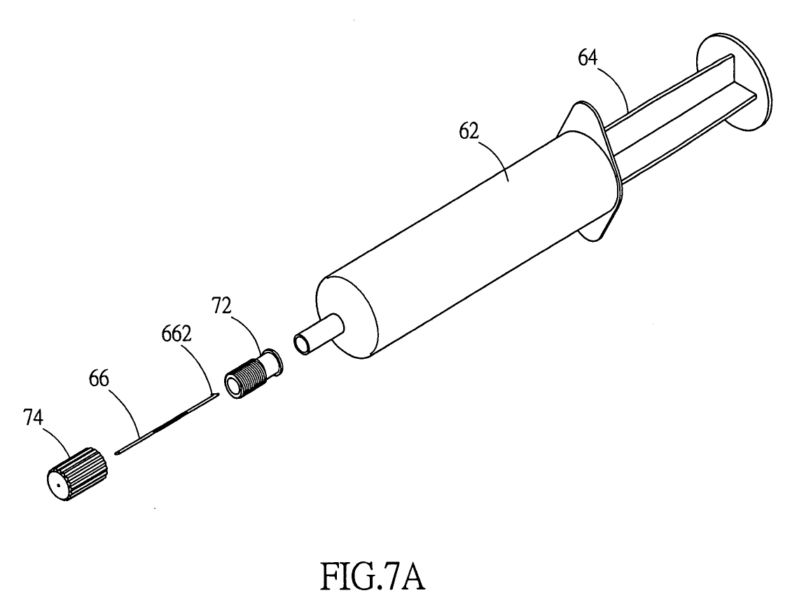

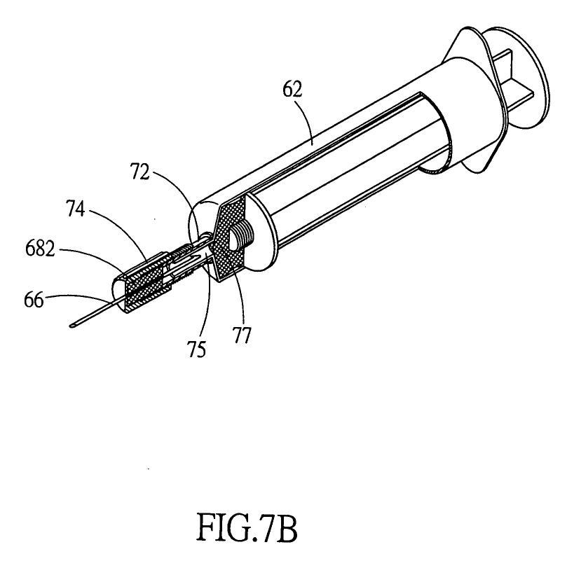

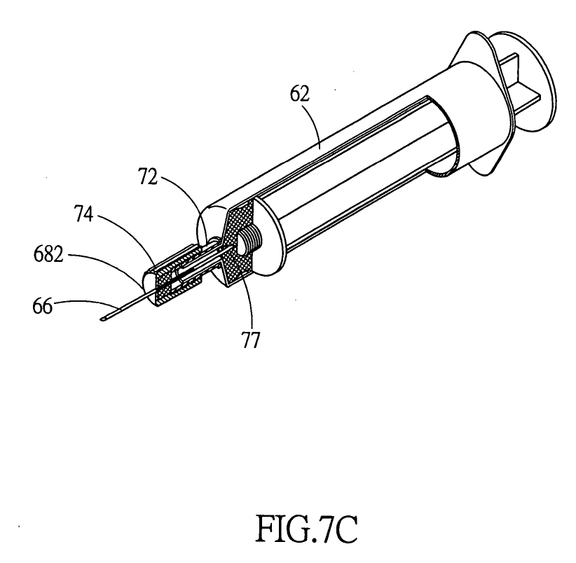

FIG. 7A through FIG. 7C are diagrams illustrating one embodiment of the present invention. The construction of the needle base 68 in the FIG. 6A can be illustrated as the FIG. 7A, which is formed by a fastening means. The fastening means can be composed of a grooved tube 72 and a nut 74. The needle 66 is configured on the nut 74. The nut 74 can comprises a first flexible material 682 for tightly holding the needle 66. Therefore the position of the hook 662 can controlled between a first position 74 and a second position 77 by the rotating the nut 74 around the grooved tube 72.

Referring to FIG. 7B and FIG. 7C, the hook 662 will not tough the second flexible material 642. That is, the second flexible material 642 does not reach the hook 662 when the first end is pushed forward. Oppositely, the hook 662 may touch the second flexible material 642 when it is in the second position 77. If the hook 662 is moved to the second position and the push pod 64 is pushed forward, the hook 662 will penetrate into the second flexible material 642. By pulling the push pod 64, the needle 66 can be pulled into the sleeve 62 after it penetrates the second flexible material 642.

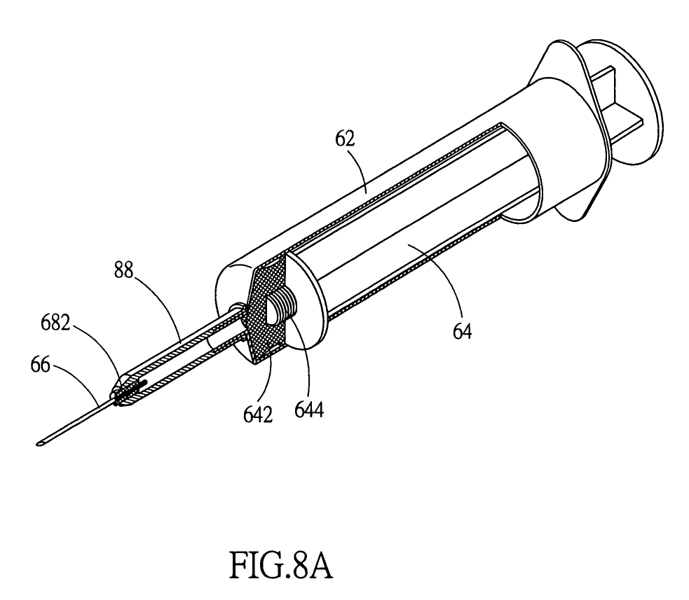

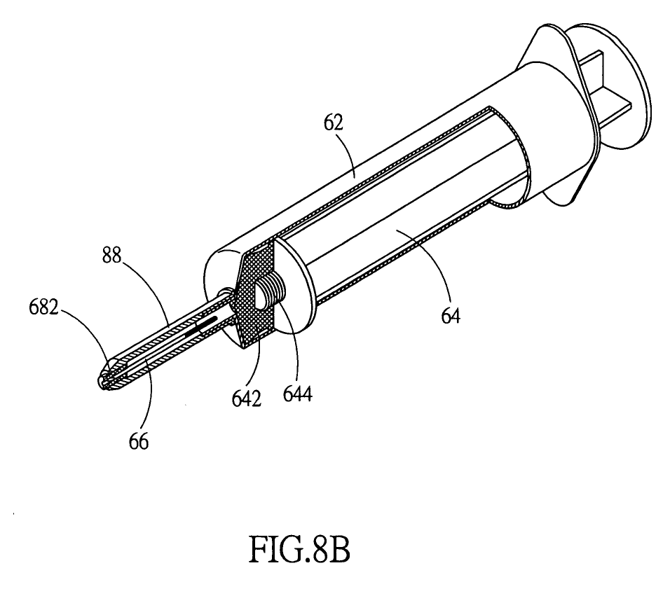

FIG. 8A is a diagram illustrating one embodiment of the present embodiment, which comprises a sleeve 62, a push rod 64, a needle 66 and a needle base 68. The sleeve 62 comprises a channel whose both ends are a first end and a second end separately, wherein the first end is connected with the needle base 68. The needle 66 is configured on the needle base 68. The needle base 68 includes a first flexible material 682 within it for tightly holding the needle 66. Thus the needle 66 penetrates the needle base 68, whereby one end of the needle 66 is outside the sleeve 62 and the other end of the needle 66 is inside the sleeve 62. The both ends of the push rod 64 are a third end and a fourth end separately. The third end is pushed into the sleeve 62 via the second end. The third end is covered with a second flexible material 642, wherein the second profile of the flexible material 642 matches the inner wall of the channel for water proof. The connection of the first end and the flexible material 642 can be a pluggable connection by a fastener means. The pluggable connection of the fastener means can be a bolt connection, a locking pawl connection, tightly holding connection and so on. Thus, the needle can be pushed into the needle base by hit the needle with a force, referring to FIG. 8B.

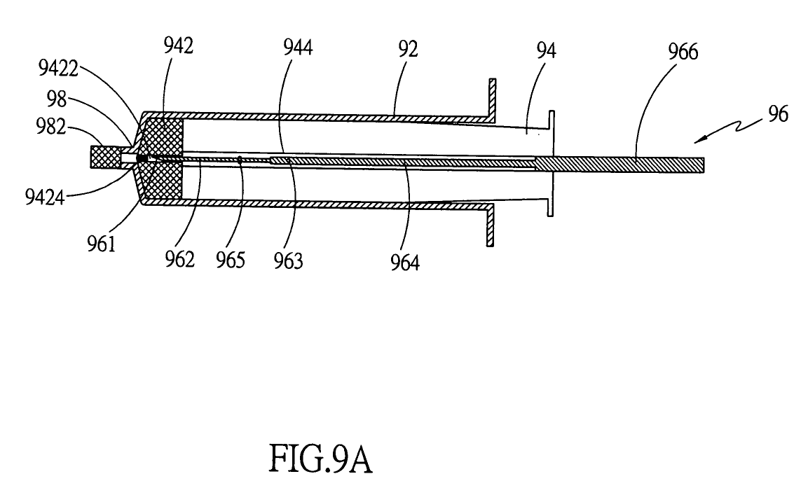

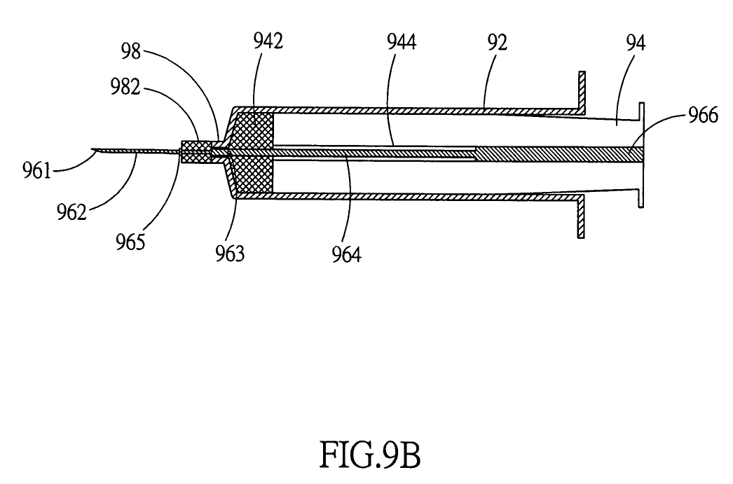

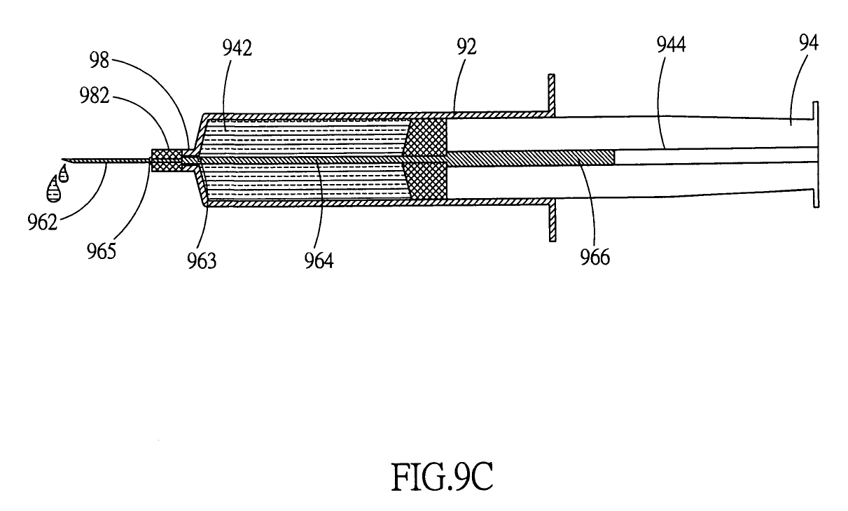

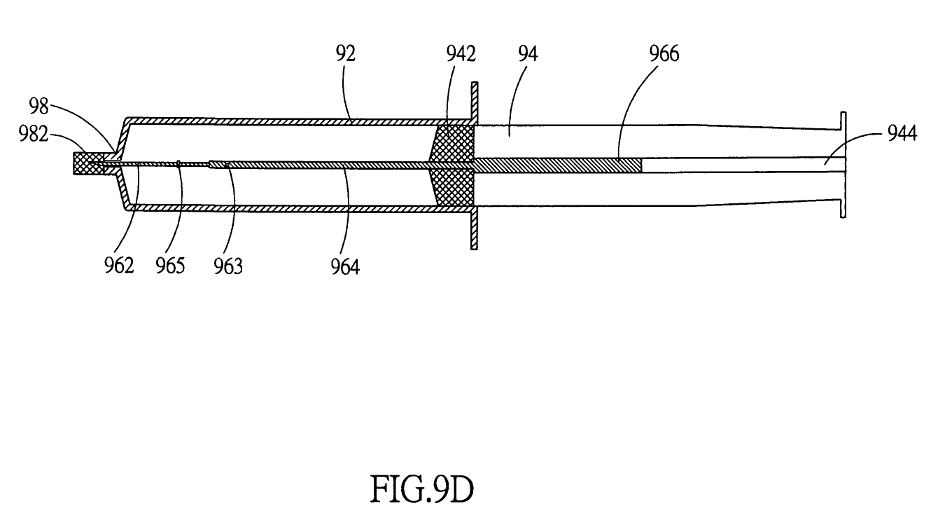

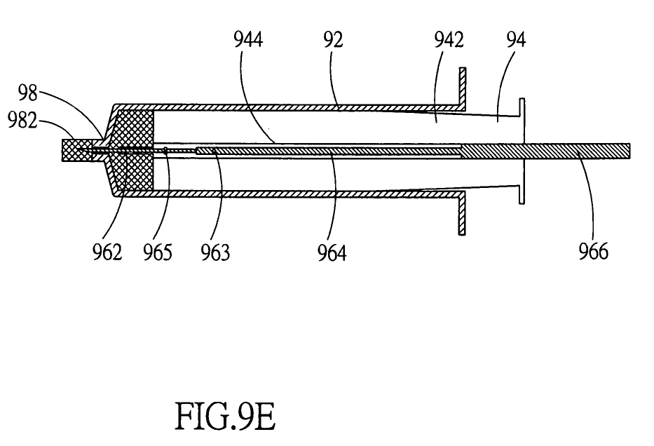

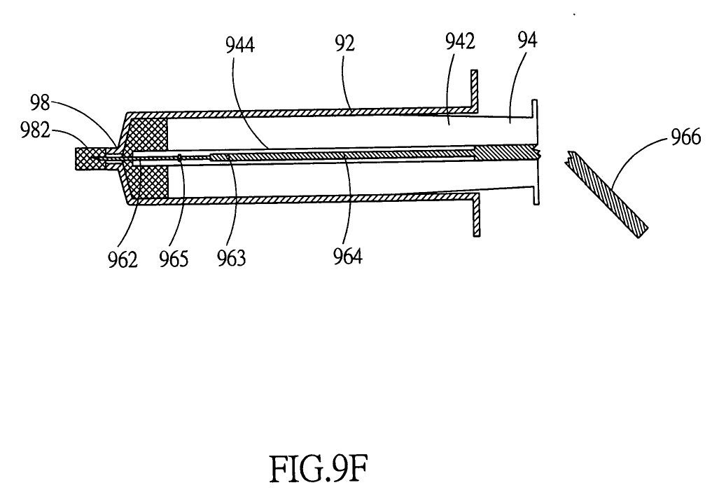

FIG. 9A is a diagram illustrating one embodiment of the present invention, which comprises a sleeve 92, a push rod 94, a needle 96 and a needle base 98. The sleeve 92 comprises a channel whose both ends are a first end and a second end, wherein the first end is connected with the needle base 98. The needle 96 is configured on the needle base 98. The needle base 98 includes an opening and a first flexible material 982 within it for tightly holding the needle 66. The first flexible material 982 is used for sealing the opening with a thickness A. The needle 96 is a three stage rod. The three stages are arranged as a fist section 962, a second section 964, and a third section 966 according to their inside diameter from small to large. The needle 96 includes a channel from the top of the first section 962 to an opening 963 which is on the surface of the needle 96. The first section comprises a prominence 965, wherein the distance between the prominence 965 and the second section 964 is larger than the thickness A. The both ends of the push rod 94 are a third end and a fourth end separately. The third end is covered with a second flexible material 942, wherein the second profile of the flexible material 942 matches the section profile of the channel for water proof. The second flexible material 942 and the push rod 94 comprise a first channel 9422 and a second channel 944 separately. The dimension of second channel 944 permits the third section 966 to pass into, and the dimension of the first channel 9422 permits the second section 964 to pass into. Therefore the second flexible material 966 can stop the third section 966 for advance passing in because of the limited dimension of the first channel. Besides, there is a third flexible material 9424 for sealing the first channel 9422.

Accordingly, the FIG. 9B illustrating the operation of the embodiment. By pushing the push rod 94, the second flexible material 942 can be pushed forward to the first end. Then push the needle 96 to penetrate the first flexible material 982, thereby the first flexible material 982 can be set between the prominent 965 and the second section 964 for fastening and holding the needle 96. In this process, the second section 964 penetrates the third flexible material 9424. Next referring to FIG. 9C, the second flexible material 942 can be pulled back by pulling the push rod 94 for sucking the drug. In the mean while, the movement of the second flexible material 942 is limited on the second section 964. That is, it is limited between the first section 962 and the third section 966. Finally, the push rod 94 can be pulled back, whereby the second section 966 is pushed by the second flexible material 942 for pulling the first section into the sleeve 92. Therefore, the top 961 of the needle can be hided behind the first flexible material, referring to FIG. 9D. The second flexible material 942 is pushed by the push rod 94 to the needle base 98, wherein the distance between the first flexible material 982 and the end of the push rod 94 within the sleeve 92 is smaller than the length of the needle 96.

Furthermore, in one embodiment illustrated in FIG. 9E, the second flexible material 942 can be pushed back to the first end by pushing the push rod 94 after the top 961 of the needle 96 is pulled behind the needle base 98, whereby a portion of the third section 966 protrudes the sleeve 92. Referring to FIG. 9F, the portion protruding the sleeve 92 can be broken.

The forgoing flexible materials can hold the penetrating object which penetrates it for water proof. The flexible materials can be plastics, foam, or so forth. The present invention does not limit it.

The connection of the first end and the flexible material 942 can be a pluggable connection by a fastener means. The pluggable connection of the fastener means can be a bolt connection, a locking pawl connection, tightly holding connection.

The foregoing description is not intended to be exhaustive or to limit the invention to the precise forms disclosed. Obvious modifications or variations are possible in light of the above teachings. In this regard, the embodiment or embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the inventions as determined by the appended claims when interpreted in accordance with the breath to which they are fairly and legally entitled.

It is understood that several modifications, changes, and substitutions are intended in the foregoing disclosure and in some instances some features of the invention will be employed without a corresponding use of other features. Accordingly, it is appropriate that the appended claims be construed broadly and in a manner consistent with the scope of the invention.

1-26. (canceled)

27. A safe injection device, comprising:
a needle, comprising at least a hook;
a needle base with a first flexible material inside it, wherein said needle is fastened by said first flexible material and protrudes from said needle base;
a sleeve with a channel that one side of said channel connects to said needle base; and
a push rod, wherein a second flexible material covers around one side of said push rod and the section profile of said side covered by said second flexible material matches the section profile of said channel for water proof.

28. A safe injection device of claim 27, wherein said hook protrudes in said channel, wherein said hook penetrates into said second flexible material by pushing said pushing rod into said channel to reach said needle base.

29. A safe injection device of claim 28, wherein hook pulls said complete needle into said channel by pulling said pushing rod after said hook penetrates into said second flexible material.

30. A safe injection device of claim 28, wherein said needle base is formed by a grooved tube and a nut, wherein said first flexible material is configured within said grooved tube.

31. A safe injection device of claim 30, wherein said needle is moved by rotating said nut into said grooved tube.

32. A safe injection device of claim 31, wherein said hook is controlled between a first position and a second position, wherein said hook can not touch said second flexible material at said first position when said second flexible material is pushed to reach said needle base, and said hook penetrates said second flexible material at said second position when said second flexible material is pushed to reach said needle base.

33. A safe injection device, comprising:
a needle;
needle base with a first flexible material inside it, wherein said needle is held by said flexible material and protrudes from said needle base, wherein the dimension of said needle base can contain the full needle;
a sleeve with a channel, wherein one side of said channel connects to said needle base; and
a pushing rod that one end of said pushing rod is covered by a second flexible material, wherein the section profile of said end covered by said flexible material matches the section profile of said channel for water proof.

34. A safe injection device, comprising:
a needle constructed by a first section, a second section, and a third section ordered by the diameter from small to large, wherein said needle includes a prominence and a injection channel from the top of the first section to an opening which is on the surface of the needle;
a sleeve with a channel;
a needle base connecting to one end of said channel, wherein said needle base comprise a second opening and a first flexible material for sealing said second opening;
a pushing rod which one end of said pushing rod is covered by a second flexible material, wherein the section profile of said end covered by said second flexible material matches the section profile of said channel of said sleeve for water proof, wherein said second flexible material and said pushing rod separately comprise a first channel and a second channel which connect to each other, wherein said second and said third section can pass into said first channel and said second channel, but said third section can not pass into said first channel because of the dimension of said first channel.

35. A safe injection device of claim 34, further comprising a third flexible material for sealing one end of said first channel.

36. A safe injection device of claim 34, wherein the distance between said prominence and said second section is larger than the width of said first flexible material.

37. A safe injection device of claim 34, wherein said second flexible material is moved within said channel to reach said needle base by pushing said push rod, in the while the distance between said first flexible material and the end of said pushing rod within said channel is smaller than the length of said needle.

38. A safe injection device of claim 34, wherein said first flexible material tightly holds the portion between said prominence and said second section of the needle.

39. A safe injection device of claim 34, wherein said needle penetrates into said first channel when said first flexible material tightly holds said needle, and said prominence is pulled to leave said first flexible material by pulling said pushing rod for using said second flexible material to pull said third section.

40. A safe injection device of claim 39, wherein the portion of said needle protruding from said sleeve can be broken after said prominence is pulled to leave said first flexible material.
